(19) United States
(12) Patent Application Publication
(10) Pub. No.: US 2017/0143967 A1
(43) Pub. Date: May 25, 2017

(54) CHOLINERGIC DEEP BRAIN STIMULATOR

(71) Applicant: Augusta University, Augusta, GA (US)

(72) Inventors: David T. Blake, JR., Augusta, GA (US); Ruifeng Liu, Augusta, GA (US); Jonathan M. Crawford, Evans, GA (US); Christos Constantinidis, Winston Salem, NC (US); Alvin V. Terry, JR., Evans, GA (US)

(21) Appl. No.: 15/361,148

(22) Filed: Nov. 25, 2016

Related U.S. Application Data
(60) Provisional application No. 62/259,696, filed on Nov. 25, 2015.

(51) Int. Cl. A61N 1/36; A61N 1/05

(52) U.S. Cl. CPC A61N 1/36082; A61N 1/0534; A61N 1/36089; A61N 1/36167

(57) ABSTRACT

A cholinergic deep brain stimulator is provided that can be used to increase brain acetylcholine levels. This increase in acetylcholine levels improves cognition and may be used to remediate disorders in which cognitive loss is a prominent symptom including Alzheimer's Disease, vascular dementia, traumatic brain injury, or drug addiction.

### CROSS REFERENCE TO RELATED APPLICATIONS

This application claims the benefit of and priority to U.S. Provisional Patent Application No. 62/259,696 filed on Nov. 25, 2015 and which is incorporated by reference in its entirety.

### STATEMENT REGARDING FEDERALLY SPONSORED RESEARCH

This invention was made with government support under 5R01MH097695 awarded by the National Institutes of Health. The government has certain rights in the invention.

### TECHNICAL FIELD

The invention is generally related to systems and methods for applying electrical stimulation to the central nervous system for treating neurological disorders or improving cognition.

### BACKGROUND

Alzheimer's Disease is a debilitating disorder that occurs in some people with age-related cognitive decline. It includes losses in memory and executive function. The current standard of care for Alzheimer's Disease is the administration of cholinesterase inhibitors like galantamine, rivastigmine, or donepezil. These inhibitors work by slowing the breakdown of acetylcholine, which enables the action of this endogenous chemical to be prolonged. The extended time of action of acetylcholine in the central nervous system is thought to improve cognition, and cholinesterase inhibitors improve some measures of cognition in young and old people alike. Because acetylcholine also acts in the peripheral nervous system, a number of peripheral side effects of the cholinesterase inhibitors limit the number of people who can tolerate them adequately to experience the cognitive benefits.

Deep brain stimulation is a generic term applied to therapies that position an electrode contact and lead inside the central nervous system, and couple it to a signal generator that is kept outside the central nervous system. This therapy has been applied most prominently in the treatment of Parkinson's Disease (U.S. Pat. No. 5,833,709). Stimulation targets in the brain's subthalamic nucleus or globus pallidus are implanted with an electrode, and high frequency stimulation, over 100 pulses per second, is applied. Parkinsonian tremor is greatly reduced using this therapy. U.S. Pat. No. 5,833,709 does not teach or suggest systems and methods for promoting or increasing acetylcholine levels in the brain or for treating Alzheimer's Disease.

Turnbull et al, in 1985, authored a manuscript on a clinical trial in one patient. The title is “Stimulation of the Basal Nucleus of Meynert in Senile Dementia of the Alzheimer's Type”. The manuscript describes intermittent stimulation unilaterally in the Nucleus Basalis with 50 Hz for 15 seconds on and 12 minutes off. No cognitive benefits were noted. Thus, Turnbull teaches that stimulation of the Basal Nucleus of Meynert is not an effective method for treating senile dementia of the Alzheimer's type.

Therefore it is an object of the invention to provide systems and methods to treat acetylcholine-related neurological disorders.

It is another object to provide systems and methods to increase brain acetylcholine levels without side effects.

### BRIEF DESCRIPTION OF THE FIGURES

FIG. 1 is a schematic diagram of one embodiment of a cholinergic deep brain stimulator.

FIG. 2A is a graph of microamps versus time (μsecs) showing a preferred stimulation pattern of current flow in one pulse. FIG. 2B is a graph of frequency (Hz) versus time (one minute) showing a preferred pulse pattern cycles each minute.

FIG. 3 is a line graph of percent correct performance versus stimulation period per minute (sec).

FIG. 4A is a graph of delay time (sec) on the left and performance (%) on the right versus Time (week) for the first monkey. FIG. 4B is a graph of delay time (sec) on the left and performance (%) on the right versus Time (week). All data shown in FIG. 4 occurs in the absence of stimulation. The vertical line in the task indicates the time point at which deep brain stimulation, on some days of the week, was initiated. In both animals, over 3 to 6 months after stimulation began, the delay period at which the target performance level was achieved became significantly elevated.

FIG. 5 is a bar graph of performance (%) versus the indicated conditions from left to right: 60 sec intermittent stimulation, Don, Both, 80 Sec Continuous stimulation, Don, Both. Don refers to the donepezil.

### SUMMARY

It has been discovered that intermittent deep brain stimulation can promote or induce the production, release, or secretion of acetylcholine in the brain. One embodiment provides a cholinergic stimulator device that includes an implantable signal generator electronically coupled to at least one implantable electrode, wherein the at least one implantable electrode is configured for implantation into, and to stimulate, at least one site that produces acetylcholine in the brain of subject in need thereof. Typically, the cholinergic stimulator device administers electrical stimulation to the brain at a rate of 10 to 80 Hz at the site of implantation for periods of 1 to 60 seconds followed by periods of no stimulation from 1 to 600 seconds wherein the stimulation period is 10% to 60% of the length of the cycle period.

In another embodiment, the cholinergic stimulator device delivers more than 200 pulses on average per minute to the implantation site.

Still another embodiment provides a cholinergic stimulator device, wherein the electrical stimulation on each cycle includes a substantial negative charge flow exceeding that induced by 100 microamps for 100 microseconds.

Typically, the one or more electrodes are implanted in one or more sites of the brain consisting of the Nucleus Basalis of Meynert, the medial septum, the diagonal band of Broca, the white matter tract in the fornix or the white matter tract from the central nucleus of the amygdala to the Nucleus Basalis or Meynert.

Yet another embodiment provides a cholinergic stimulator device, wherein the stimulation pattern has 60 pulses per second stimulation for half as long a period of time as the no stimulation period resulting in 1200 pulses per minute.

In other embodiments, the cholinergic stimulator device delivers 60 pulses per second for 20 seconds, with no stimulation for an additional 40 seconds. This cycle is repeated as necessary for example, for the entire day, or for the entire length of time in which behavioral training is applied.

Another cholinergic stimulator device delivers 1200 pulses in 10 seconds per minute, 20 seconds per minute, 40 seconds per minute, or 60 seconds per minute.

Methods of using the cholinergic stimulator devices are also provided. One embodiment provides a method for increasing acetylcholine in the brain of a subject in need thereof, comprising applying deep brain stimulation with any one of the disclosed cholinergic stimulator devices. The method can incorporate a stimulation that delivers 60 pulses per second for 20 seconds, with no stimulation for an additional 40 seconds. Stimulation cycles are applied over a period of days, weeks, months, or years.

In another embodiment, the stimulation delivers 1200 pulses in 10 seconds per minute, 20 seconds per minute, or 40 seconds per minute.

The disclosed methods can be performed on a subject that has or is believed to have Alzheimer's Disease, vascular dementia, traumatic brain injury, or drug addiction.

In certain embodiments, the methods can be combined with a second treatment including for example combining the brain stimulation with practice at tasks using working memory, vigilance, or set shifting behavioral tasks.

Yet another embodiment provides a method for treating Alzheimer's Disease, vascular dementia, traumatic brain injury, or drug addiction by stimulating the brain of the subject with the disclosed cholinergic stimulator device to increase production or release of acetylcholine in the brain of the subject.

### DETAILED DESCRIPTION

### I. Definitions

Unless defined otherwise, all technical and scientific terms used herein have the same meanings as commonly understood by one of skill in the art to which the disclosed invention belongs. In particular, the following terms and phrases have the following meaning.

The use of the terms “a”, “an”, “the” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “individual”, “host”, “subject”, and “patient” are used interchangeably herein, and refer to a mammal, including, but not limited to, primates, for example, human beings, as well as rodents, such as mice and rats, and other laboratory animals.

The term “intermittent stimulation’ refers to stimulation that occurs for some subperiod followed by a subperiod with no stimulation, after which the stimulation cycle repeats. Within the stimulation subperiod, a substantial negative charge exceeding 100 microamps for 100 microseconds is delivered in each cycle of the stimulation frequency. Within the no stimulation subperiod, no substantial negative charges are delivered.

The term “continuous stimulation” refers to stimulation that occurs at a fixed frequency without interruptions. In each cycle of this fixed frequency, a substantial negative charge exceeding 100 microamps for 100 microseconds is delivered.

The term “stimulation cycle” refers to the subperiod of stimulation in intermittent stimulation which includes on subperiod of stimulation and one subperiod of no stimulation.

The term “working memory task” refers to a task that involves presenting the subject with information followed by a period of seconds to minutes after which the subject is tested on recall of that information. A canonical example would be “list recall”.

The term “vigilance task” refers to a task that involves the subject continuously monitoring inputs and responding whenever target inputs are presented over a period of minutes. A canonical example would be the “continuous performance task”.

The term “set shifting task” refers to a task in which the subject is required to sort inputs according to sorting rules that must be inferred from feedback on appropriate sorting, and in which the sorting rules are periodically changes to require the subject to infer or deduce the new sorting rules. A canonical example would be the “Wisconsin Card Sort” task.

As used herein, the term “treatment regimen” refers to a treatment of a disease or a method for achieving a desired physiological change, such as increased acetylcholine production or secretion, to produce a physiological change. On example is the administration of an electric current stimulus to a target region of the brain of a subject. The electric stimulus can be administered in separate distinct bursts for a period time separated by a finite period of time and repeated as needed.

### II. Cholinergic Deep Brain Stimulator

Cholinergic deep brain stimulator systems and methods of use are described. In one embodiment, the stimulator system electrically activates target nuclei. In one embodiment, activating target nuclei improves cognition, increases production or release of acetylcholine, or both. In still another embodiment, the cognitive improvement relieves symptoms of disorders associated with a loss of cognition, for example Alzheimer's Disease.

The natural source of acetylcholine for the cerebral cortex, which is the region thought important for the cognitive benefits of cholinesterase inhibitors, is one of several nuclei. The Nucleus Basalis of Meynert is the source of acetylcholine for neocortex. It receives a strong input from the central nucleus of the amygdala. The medial septum, and Diagonal Band of Broca are forebrain nuclei that release acetylcholine to the other telencephalon regions, the medial temporal lobe and hippocampus. Portions of these projections target the hippocampus through the white matter track called the fornix. The endogenous source of acetylcholine to the cerebral cortex comes through these nuclei and pathways.

One embodiment provides a method that includes positioning the electrode lead of an exemplary deep brain stimulation system into one of these targets: Nucleus Basalis of Meynert, projection from the central nucleus of the amygdala to the Nucleus Basalis of Meynert, Medial Septum, Diagonal Band of Broca, or fornix, and stimulating the target release more acetylcholine.

In another embodiment, the stimulation must be intermittent, with significant breaks in periods of stimulation. An exemplary embodiment stimulates for 20 seconds, with at least a 40 second period in between each period of stimulation. Intermittent stimulation may be applied for a time period as short as an hour, or all the time day and night. Additionally, more pulses per minute, up to at least 1000 pulses, are required to achieve optimal effects.

In one embodiment, the disclosed cholinergic deep brain stimulator systems are made of materials suitable for implantation into the human body. For example, the materials do not cause or promote an inflammatory response, an allergic response, or immune response. Thus, in some embodiments, the systems are made of materials that are one or more of the following: non-immunogenic, hypoallergenic, non-pyrogenic, and sterile.

A. System Components

### 1. Electrodes

An exemplary cholinergic deep brain stimulator includes one or more electrodes 110. In one embodiment, the electrodes 110 were custom manufactured in the laboratory based on published specifications (McCairn, K. W., ID-ORCID: http://orcid.org/0000-0002-8546-4095 & Turner, R. S. Pallidal stimulation suppresses pathological dysrhythmia in the parkinsonian motor cortex. J. Neurophysiol. 113:2537-2548 (2015); McCairn, K. W. & Turner, R. S. J. Neurophysiol. Φ, 1941-1960 (2009) both of which are incorporated herein in their entireties). Conductors were 50 gm Pt/Ir Teflon wire (A-M systems, Seattle, Wash.) embedded within a 30 ga. hypodermic tube, which was encased in a 28 ga. polyimide sheath. The wire extended from the neural end of the sheath by roughly 1 mm, and the last 0.7 mm of insulation were stripped to achieve impedances of 5-10 kOhm at 1 kHz. The far end of the electrode was soldered to an extracranial connector.

The electrodes and leads are also commercially available.

The electrode assembly can be implanted transdurally, via a chronic recording chamber using a transdural protective guide cannula (26 ga.) and stylus mounted in the microdrive. Upon reaching the target location for implantation, the guide tube was withdrawn, and the electrode depth was fixed in the recording chamber before the stylus was removed. Methods for implanting electrodes into the brain are known in the art.

### 2. Pulse Generators

The electrodes 110 are electronically coupled to pulse signal generator 115. Pulse signal generators, including implantable pulse signal generators have been extensively used in treating movement disorders with an extensive prior art dating to the 1980s (e.g., Kumar, R. et. al., “Double-blind evaluation of subthalamic nucleus deep brain stimulation in advanced Parkinson's disease”, Neurology, 51:850-855, 1988) and are commercially available. In a preferred embodiment, the pulse signal generator is an implantable pulse signal generator. An exemplary implantable pulse signal generator is the Precision Novi™ Implantable Pulse Generator available from Boston Scientific. The pulse generator can be a multi-channel, multi-electrode device. The pulse generator can be programmable and contain a power source, for example a battery. The pulse generator can be remotely controlled (i.e, wirelessly controlled) and remotely programmed to change the frequency, duration, duty cycle, and delay interval of the electric pulses.

Exemplary stimulation patterns that can be programmed into the pulse signal generator include, but are not limited to periods of stimulation interleaved with periods of no stimulation. In the preferred implementation, the stimulation delivers 60 pulses per second for 20 seconds, with no stimulation for an additional 40 seconds. In another embodiment, 1200 pulses are delivered in 10 seconds per minute, 20 seconds per minute, or 40 seconds per minute.

In still another embodiment, system 100 at a rate of 10 to 80 Hz for periods of 0.1 to 60 seconds followed by periods of no stimulation from 0.05 to 600 seconds and wherein the stimulation period is 10% to 200% of the length of the no stimulation period and more than 200 pulses are stimulated on average per minute. The electrical stimulation on each cycle typically includes a substantial negative charge flow exceeding that induced by 100 microamps for 100 microseconds.

### B. Exemplary Embodiments

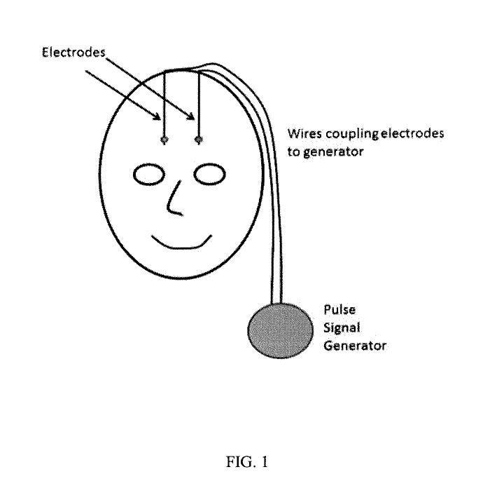

FIG. 1 is a schematic view of an exemplary cholinergic deep brain stimulator system 100. System 100 includes implantable electrodes 105 that are positioned in one or more target areas in the central nervous system of a subject or patient. The electrodes 105 are electrically coupled to the pulse signal generator 110. In a preferred embodiment, electrodes 105 and pulse generator 110 are both implanted into the body of a subject or patient.

In one embodiment one or more electrodes 105 are positioned inside the brain of a subject in need of neurostimulation. System 100 generates and applies a stimulus, for example an electrical stimulus, to the targeted area of the brain to activate different brain nuclei than are used in treating movement disorders like Parkinson's and dystonia. For example, one embodiment targets the Nucleus Basalis of Meynert, the Medial Septum, the Diagonal Band of Broca, combinations thereof or their direct inputs or outputs for stimulation. These targets are neuronal nuclei and pathways that release acetylcholine. In the preferred implementation, electrodes are positioned bilaterally in the Nucleus Basalis of Meynert.

### III. Methods of Treatment

System 100 can used to induce the formation of acetylcholine in the brain of a subject in need thereof. The induction or promotion of acetylcholine formation in the brain can be used to treated acetylcholine-related neural pathologies. Exemplary acetylcholine-related neural pathologies that can be treated with the disclosed systems and methods include, but are not limited Alzheimer's Disease, Alzheimer's related-dementia, memory loss, vascular dementia, traumatic brain injury, or drug addiction.

One embodiment provides a method for increasing levels of acetylcholine in the brain of a subject in need thereof by administering an electrical stimulus to a target area of the brain, wherein the electrical stimulus has a signal pattern of 60 pulses per second for 20 seconds, with no stimulation for an additional 40 seconds. In another embodiment, 1200 pulses are delivered in 10 seconds per minute, 20 seconds per minute, 40 seconds per minute, or 60 seconds per minute.

The disclosed methods can be performed on a subject that has or is believed to have Alzheimer's Disease, vascular dementia, traumatic brain injury, or drug addiction.

Yet another embodiment provides a method for treating loss of cognition, Alzheimer's Disease, vascular dementia, traumatic brain injury, or drug addiction by stimulating the brain of the subject with the disclosed cholinergic stimulator device to increase production or release of acetylcholine in the brain of the subject.

In certain embodiments, the methods can be combined with a second therapeutic agent or second treatment including for example combining the brain stimulation with practice at tasks using working memory, vigilance, or set shifting behavioral tasks.

The disclosed methods can be combined with existing methods of treating Alzheimer's Disease other than cholinesterase inhibitors. Existing treatments for Alzheimer's Disease include but are not limited to Memantine.

### EXAMPLES

### Example 1

### Intermittent Stimulation of the Nucleus Basalis of Meynert (NBM)Results is Superior Performance

Materials and Methods

Behavioral Task

All behavioral software was custom programmed, and based on prior work (Terry Jr., A., Psychopharmacol. (Berl) 233:761-771 (2016)). The behavioral task, shown in FIG. 1, initiates with a colored square cue in the center-top part of the touchscreen. Upon touching the square, the cue disappears, and the screen remains blank for the delay period. In the match phase, two colored squares are presented in the lower right, and lower left, of the touchscreen. One of the two squares, randomized in location, has the same color as the cue square. Correctly touching the target square results in delivery of food slurry reward. Percent correct and reaction times are recorded. Animals required several months of daily training to achieve stable performance at the task.

Two young adult Rhesus monkeys were pre-trained in a delayed match to sample task. Animals typically performed behavioral trials for 500-800 rewards each day.

Surgery and Deep Brain Stimulation

Surgery was performed in a sterile surgical suite under isoflurane anesthesia monitored by a clinical veterinarian. Stereotaxic measurements were made to target implantation at 8 mm lateral, 16 mm anterior interaural, and 29 mm below the cortical surface in a vertical penetration. A skin flap was performed, and two holes were drilled above the location of each implant. A small titanium chamber was mounted on the cranium, and the electrodes were lowered to depth. Silicone was poured into the chamber, and the electrode depth was fixed. The skin was sutured closed, and connectors enabled repetitive access to the electrode.

All stimulation was performed using biphasic, charge-balanced pulses with 100 μs per phase, 200 μA in amplitude, negative-first. Continuous stimulation was performed from 10 pulses per second to 120. Intermittent stimulation was initially applied at 60 pulses per second only in-between trials, or only during trials, which were roughly 3 and 5 second periods. Thereafter, experiments standardized on a 1 minute cycle length, and stimulated for 20 seconds at 60 pulses per second, followed by 40 seconds with no stimulation. In these one minute cycle stimulation experiments, no attempt was made to synchronize the stimulation with the behavioral trials.

Electrodes were custom manufactured in our laboratory based on published specifications (McCairn, K. W., ID-ORCID: http://orcid.org/0000-0002-8546-4095 & Turner, R. S. Pallidal stimulation suppresses pathological dysrhythmia in the parkinsonian motor cortex. J. Neurophysiol. 113:2537-2548 (2015); McCairn, K. W. & Turner, R. S. J. Neurophysiol. 101, 1941-1960 (2009)). Conductors were 50 μm Pt/Ir Teflon wire (A-M systems, Seattle, Wash.) embedded within a 30 ga. hypodermic tube, which was encased in a 28 ga. polyimide sheath. The wire extended from the neural end of the sheath by roughly 1 mm, and the last 0.7 mm of insulation were stripped to achieve impedances of 5-10 kOhm at 1 kHz. The far end of the electrode was soldered to an extracranial connector.

The electrode assembly was implanted transdurally, via the chronic recording chamber using a transdural protective guide cannula (26 ga.) and stylus mounted in the microdrive. Upon reaching the target location for implantation, the guide tube was withdrawn, and the electrode depth was fixed in the recording chamber before the stylus was removed.

Local Field Potential Measurements

The local field potential for intermittent stimulation was measured in a six second period after 20 seconds of stimulation at 60 Hz, and was compared to the resting condition after five minutes with no stimulation. The continuous stimulation local field potential was measured in six seconds after five minutes of continuous stimulation at 80 Hz, and the resting condition was the local field potential after no stimulation for five minutes. Both sets of local field potentials were collected in interleaved data during the same sessions. The recording reference was the animal headpost, which was positioned extracranially roughly at vertex. Signals were highpass filtered at 1 Hz to remove stimulation artifacts.

Donepezil Administration

Donepezil was given via I.M. administration on the mornings in which data was collected. The rationale for the doses selected for donepezil was based on previous behavioral and functional brain imaging data (dose range 0.05-0.250 mg/kg) in rhesus monkeys.

Data from days without donepezil were at least 48 hours later than the last administration. Animals administered doses higher than 200 μg/kg would not perform an adequate number of trials for food reinforcement to generate performance data. One dose was tested per week. Each week, control performance was evaluated. Then, performance under the intermittent stimulation alone, donepezil alone, and donepezil+stimulation were evaluated. Similar weekly plans were used for continuous stimulation.

All animal studies comply with the Guide for the Care and Use of Animals, 8th Edition, and were approved by the IACUC at Augusta University.

d'Calculation

The signal detection theoretic d′ is the z-score difference between the hit rate and false alann rate. In our two alternative forced choice framework, d′ is calculated in Matlab (Mathworks, Natick, Mass.) as √2* norminv(Hit Rate).

Binomial Statistical Model

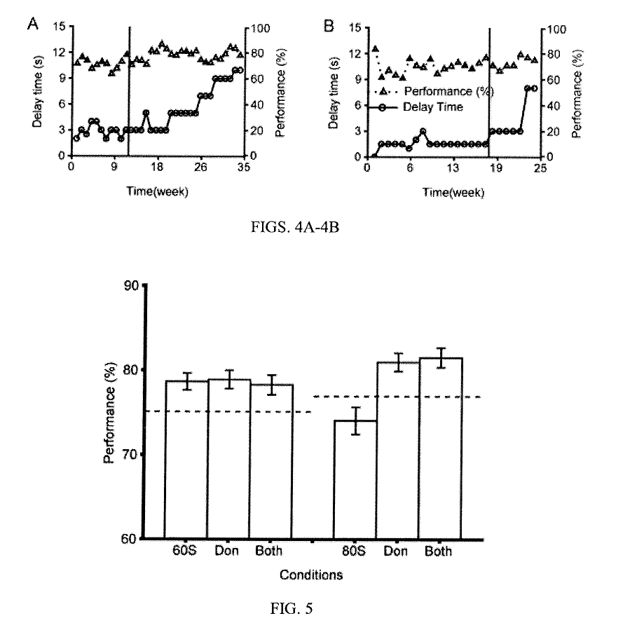

In the long-term delay change data (FIG. 4C-D), animals performed 3000 trials weekly at a performance level averaging 78% correct. The delay curves in FIG. 3 for the control condition have 5.4 and 5.1 percent improvements if the delay is changed by a factor of two from the right-most datapoint which is close to 78%. We conservatively round this down to 5 percent which would be associated with a duration change slightly smaller than a halving. Then, the two-tailed probabilities are calculated that a binomial process with a fraction correct of 0.78 over 3000 trials could result in more correct trials than a process with a fraction correct 5 percent higher or lower. This probability is less than 10-5. If a threshold is set that the duration doubles and the percent correct stays at 78% or higher, then the working memory performance is significantly better. The implication of the binomial model is that if an animal performs at 78% correct for duration 2X, then it would be expected to perform at 83%, or higher, at duration X, and that this performance would be significantly better than its pre-stimulation performance of 78% at duration X.

Results

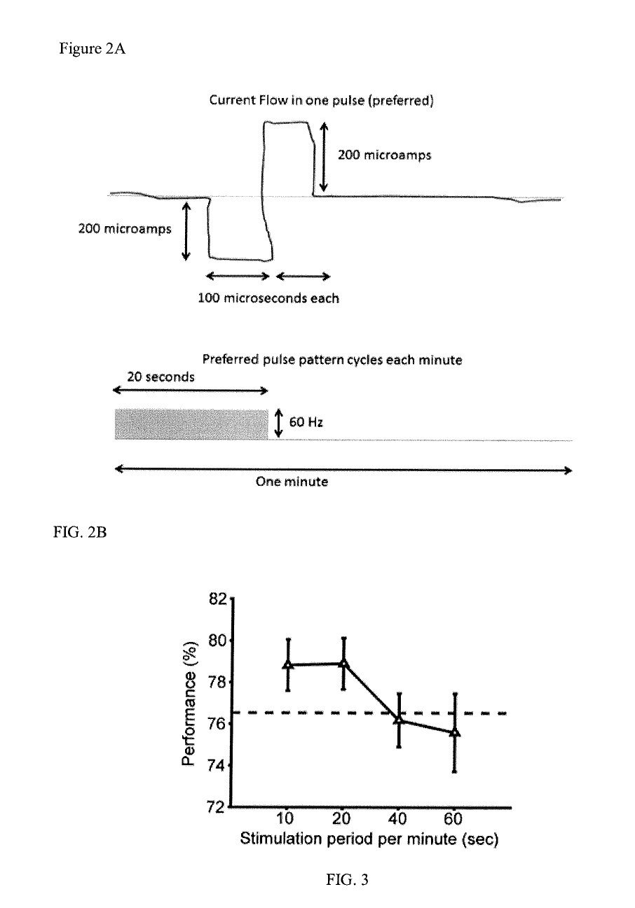

The hypothesis that stimulation of the Nucleus Basalis of Meynert (NBM) alters working memory performance was tested. Animals were delivered continuous stimulation throughout blocks of trials. However, continuous stimulation always impaired performance, and effects were larger at higher stimulation rates. Results for continuous stimulation at 80 Hz are statistically significant, (binomial tests on 80 Hz stimulation, Animal One: n=800, p<0.0001; Animal Two: n=1000, p<0.001), as shown in FIG. 2A. Stimulation was used in blocks of 100 trials interleaved with blocks of 100 trials without stimulation.

In an effort to determine if stimulation during a particular interval of the task was disrupting working memory, the task was altered to stimulate only during the inter-trial period, or to stimulate only during trials. Unexpectedly, either condition resulted in significantly improved performance over normal performance (binomial test, n=500, p<0.05). Performance benefits were thereafter tested using intermittent stimulation with 20 seconds of pulses at a rate of 60 pulses per second, and 40 seconds in between stimulation periods (FIG. 2B).

FIGS. 2A and 2B show a preferred stimulation pattern. In this example, the stimulation pattern contains periods of stimulation interleaved with periods of no stimulation. In the preferred implementation, the stimulation delivers 60 pulses per second for 20 seconds, with no stimulation for an additional 40 seconds. The practice of interleaving periods of stimulation and no stimulation is referred to as intermittent stimulation. In the examples given in FIG. 2 through 5, intermittent stimulation was applied for 1 to 6 hours per day, 1 to 5 days per week, over a period of months. In practice,effects will depend on stimulation being applied at least one hour per week over a period of months, and may be applied more often.

### Example 2

### Stimulation over Different Duty Cycles

To clarify why these two conditions resulted in behavioral improvement or impairment, two further experiments were conducted. First, animals were stimulated with 1200 pulses per minute over different duty cycles. Animals were tested with 1200 pulses delivered in 10 seconds per minute, 20 seconds per minute, 40 seconds per minute, or 60 seconds. A two way ANOVA found a significant effect of these conditions on performance (test for animal number and condition, F=12.92 for condition, p<0.02, F=1.4 for animal p>0.1). Post hoc comparisons show that performance is significantly better at 10 or 20 second periods than the 60 second period (binomial statistic, roughly 1050 trials each, Bonferroni corrected p<0.005) The second experiment stimulated with 80 pulses per second for 10, 20, 40, or 60 seconds per minute. A two-way ANOVA found a significant effect of condition and animal (F=14.28 for condition p<0.02, F=7.55 for animal p<0.04). The highest performance occurred for the 20 second stimulation condition which was significantly better than the 60 second period in post hoc tests (binomial statistic, roughly 1350 trials each, Bonferroni corrected p<0.005). The significant ANOVA finding on the animal variable occurred as Animal One averaged higher performance in the 80 Hz stimulation conditions than Animal Two despite nearly identical control performance rates.

The working memory task can be deconstructed into the process of encoding the memory, the process of maintaining the memory, and the process of retrieving the memory. Maintaining the memory is a process by which activity in the prefrontal cortex and areas of the association cortex connected to it maintain elevated action potential rates during the delay period. To investigate the dependency of the stimulation effects on maintenance, the effects were determined as a function of the delay period.

FIG. 3 shows behavioral results from prototype testing in Rhesus monkeys (conducted under NIH funding and the approval of the IACUC at Augusta University). In the four displayed conditions, 1200 pulses per minute are delivered. The first case delivers the 1200 pulses in 10 seconds at 120 Hz. The second case is 60 Hz for 20 seconds. The third case is 30 Hz for 40 seconds. The last case is continuous stimulation. The dashed line indicates the performance level with no stimulation. Two monkeys are performing a delayed match to sample with two stimuli. Chance performance is 50% correct, and 75% correct is halfway between chance and perfect performance. The delay is animal specific and set in this study to maintain performance between 75 and 80% correct. The addition of 1200 pulse per minute stimulation, if delivered in 10 or 20 seconds, improves performance. If stimulation is continuous (60 second data point), no cognitive improvement occurs. All major deep brain stimulation for the treatment of movement disorders uses continuous stimulation. This is the first report that intermittent stimulation patterns described herein have cognitive benefits.

The performance change, expressed as change in percent correct, was larger for longer delays than for shorter delays. These percent change differences were converted to changes in signal size using signal detection theory metrics, and then the change in signal detectability is larger for shorter delays. In any case, using either signal detectability metrics or percent correct, no statistically significant changes in stimulation effect were found as a function of delay length.

### Example 3

### Onset of Stimulation Effects

Insight into the speed with which these effects occur after stimulation begins was determined by analyzing the performance compared to the trial position within the trial block. If effects appear slowly, then an intermittent stimulation block of 50 trials, which requires roughly 15 minutes to complete, should have higher performance late in the block compared to earlier in the block. Similarly, if the effects decrease slowly once stimulation stops, the no-stimulation blocks of 50 trials should have lower performance later in the block. No significant trends based on position within the blocks were found, which supports effects occurring within 3 minutes, or the average time to complete 10 trials. In addition, the behavioral impact of stimulation for an entire day is indistinguishable from its impact in an alternating block design. Therefore, the effects onset rapidly enough that behavioral modulation is present within at most 3 minutes, and that short term cognitive effects cease within at most 3 minutes after stimulation ends. Note that in FIG. 5, it is demonstrated that long term effects occur within months, and that these effects are caused by stimulation, but occur even in its absence.

The duration of the working memory delay over which the Animal One could maintain a performance of 75-85% correct in the absence of stimulation increased five-fold within five months after the beginning of stimulation of Nucleus Basalis. These delays were reconstructed, as a function of time. Each week represents approximately 3000 trials. To ensure that this improvement was not the result of accumulated exposure to the task, regardless of stimulation, the next two animals were allowed to reach asymptotic performance in the delayed-match-to sample task, over 10 and 17 weeks, before we initiated the stimulation experiments. The ensuing initiation of stimulation led to a three- and five-fold increase in working memory delay duration.

FIGS. 4A and 4B show the long-term changes that occur once deep brain stimulation of the Nucleus Basalis of Meynert begins. Results from one monkey are shown in FIG. 4A, and a second monkey in FIG. 4B. In this working memory task, the performance decreases as the delay period increases, or as the monkey is required to remember the cue for a longer period. The delays were changed weekly for each animal to ensure performance in the absence of stimulation was between 75 and 80% correct. All data shown in FIGS. 4A and 4B occur in the absence of stimulation. The left abscissa shows the delay used that week, and the right abscissa shows the percent correct for that week. The vertical line in the task indicates the time point at which deep brain stimulation, on some days of the week, was initiated. In both animals, over 3 to 6 months after stimulation began, the delay period at which the target performance level was achieved became significantly elevated.

FIG. 5 shows the result of combining either continuous stimulation with the use of a cholinesterase inhibitor donepezil, or of combining the preferred implementation of the intermittent stimulation with the cholinesterase inhibitor. Acetylcholine, once released, is cleaved into acetate and choline which are inactive. The choline is taken back into the neuron, and converted to acetylcholine by the enzyme choline acetyltransferase. Once converted, acetylcholine can be cellularly prepared for release again. Donepezil reduces the cleavage of acetylcholine into acetate and choline, which prolongs the action of each acetylcholine molecule after release. If the intermittent stimulation increased the net effect of acetylcholine, it would be expected to interact with the cognitive changes induced by donepezil. As seen on the left in FIG. 5, the donepezil and the deep brain intermittent stimulation (60S) each improved behavioral performance. If the two treatments are combined, no further benefit is seen because both work by boosting the same mechanism. The dashed line shows the performance on days without stimulation or donepezil. As seen on the right in FIG. 5, the continuous stimulation (80S) impairs behavioral performance, and this impairment is turned into a behavioral improvement when combined with donepezil. The continuous stimulation reduces brain acetylcholine levels.

We claim:

1. A cholinergic stimulator device comprising:
an implantable signal generator;
at least one implantable electrode electrically coupled to the implantable signal generator, wherein the at least one implantable electrode is configured for implantation into, and to stimulate, at least one site that produces acetylcholine in the brain of subject in need thereof;
wherein the cholinergic stimulator devices administers electrical stimulation to the brain at a rate of 10 to 80 Hz for periods of 0.1 to 60 seconds followed by periods of no stimulation from 0.05 to 600 seconds and wherein the stimulation period is 10% to 200% of the length of the no stimulation period to induce the production or release of acetylcholine in the brain.

2. The cholinergic stimulator device of claim 1, wherein more than 200 pulses are generated on average per minute.

3. The cholinergic stimulator device of claim 1 or claim 2, wherein the electrical stimulation on each cycle of the stimulation frequency includes a substantial negative charge flow exceeding that induced by 100 microamps for 100 microseconds.

4. The cholinergic stimulator device of claim 1, wherein the one or more electrodes are implanted in one or more sites of the brain consisting of the Nucleus Basalis of Meynert, the medial septum, the diagonal band of Broca, the white matter tract in the fornix or the white matter tract from the central nucleus of the amygdala to the Nucleus Basalis of Meynert.

5. The cholinergic stimulator device of claim 4, wherein the one or more electrodes are implanted in the Nucleus Basalis of Meynert.

6. The cholinergic stimulator device of claim 1, wherein the stimulation pattern applies 60 pulses per second stimulation for half as long a period of time as the no stimulation period resulting in 1200 pulses per minute.

7. The cholinergic stimulator device of claim 1, wherein the stimulation delivers 60 pulses per second for 20 seconds, with no stimulation for an additional 40 seconds.

8. The cholinergic stimulator device of claim 1, wherein the stimulation delivers 1200 pulses in 10 seconds per minute, 20 seconds per minute, or 40 seconds per minute.

9. A method for increasing acetylcholine in the brain of a subject in need thereof, comprising applying deep brain stimulation with the device of claim 1.

10. The method of claim 9, wherein the stimulation delivers 60 pulses per second for 20 seconds, with no stimulation for an additional 40 seconds.

11. The method of claim 9, wherein stimulation cycles are applied over a period of days, weeks, months, or years.

12. The method of claim 9, wherein the stimulation delivers 1200 pulses in 10 seconds per minute, 20 seconds per minute, or 40 seconds per minute

13. The method of claim 9, wherein the subject has or is believed to have Alzheimer's Disease, vascular dementia, traumatic brain injury, or drug addiction.

14. The method of claim 9, wherein the brain stimulation is combined with practice at tasks using working memory, vigilance, or set shifting behavioral tasks.

15. The method of claim 9, wherein the one or more electrodes are implanted in one or more sites of the brain consisting of the Nucleus Basalis of Meynert, the medial septum, the diagonal band of Broca, the white matter tract in the fornix or the white matter tract from the central nucleus of the amygdala to the Nucleus Basalis or Meynert.

16. A method for increasing cognition in a subject comprising stimulating the brain of the subject with cholinergic stimulator device of claim to increase production or release of acetylcholine in the brain of the subject.

17. The method of claim 16, wherein the subject has or is believed to have Alzheimer's Disease, vascular dementia, traumatic brain injury, or drug addiction.

18. A method for treating Alzheimer's Disease in a subject in need thereof, comprising stimulating the brain of the subject with cholinergic stimulator device of claim to increase production or release of acetylcholine in the brain of the subject.
